(19) United States
(12) Patent Application Publication
(10) Pub. No.: US 2018/0200402 A1
(43) Pub. Date: Jul. 19, 2018

(54) FIXING TOOL

(71) Applicant: TERUMO KABUSHIKI KAISHA, Tokyo (JP)

(72) Inventors: Ryo OKAMURA, Fujinomiya-shi (JP); Tatsunori FUJII, Bear, DE (US); Miho KAI, Hadano-shi (JP); Takashi AOKI, Nakakoma-gun (JP)

(73) Assignee: TERUMO KABUSHIKI KAISHA, Tokyo (JP)

(21) Appl. No.: 15/866,971

(22) Filed: Jan. 10, 2018

(30) Foreign Application Priority Data
Jan. 19, 2017 (JP) 2017-007784

(51) Int. Cl. A61L 15/14; A61L 15/10; A61F 5/05

(52) U.S. Cl. CPC A61L 15/14; A61B 17/6408; A61F 5/05; A61L 15/10

(57) ABSTRACT

A fixing tool is disclosed, which can help reduce a possibility that heat may be transferred to a user when a state is changed in a material of a member for fixing a predetermined site of a human body. The fixing tool has a deformation portion that is configured to be deformable, that at least partially covers an outer periphery of any site of a limb, a trunk, a neck, and a head, and that is deformed to align with the site through the deformation, and a heat insulating material that is disposed in a portion corresponding to at least the site on an outside surface of the deformation portion.

### CROSS-REFERENCES TO RELATED APPLICATIONS

This application claims priority to Japanese Application No. 2017-007784 filed on Jan. 19, 2017, the entire content of which is incorporated herein by reference.

### TECHNICAL FIELD

The present disclosure relates to a fixing tool.

### BACKGROUND DISCUSSION

When a medical procedure is performed, for example, on a leg of a patient in hospital, the leg serving as a target lesion of the patient may need to be fixed. When the target lesion is fixed, the leg becomes numb depending on a fixing method. In some cases, there is also a possibility of neurological damage. Therefore, in order to prevent the above-described disadvantage when the medical procedure is performed, the leg of the patient is fixed using a tool. For example, Japanese Patent No. 5955054 discloses a fixing tool as follows. A solidifying agent such as a liquid contained in a bag is mixed with a substance for initiating solidification of a metal piece or the like so as to solidify the solidifying agent. In this manner, a site of the leg can be fixed using the fixing tool.

However, when the solidifying agent is deformed to align with a shape of a predetermined site of a human body as disclosed in Japanese Patent No. 5955054, the solidifying agent may generate heat when the liquid is changed to a solid or the like. Consequently, there is a possibility that a user may feel discomfort.

### SUMMARY

The present disclosure aims to provide a fixing tool which can help reduce a possibility that a user may feel discomfort due to a state change in a material of a member for fixing a predetermined site of a human body.

A fixing tool according to the present disclosure has a deformation portion and a cover. The deformation portion is brought into a deformable state by mixing two or more different materials with each other, and is cured in a state where the deformation portion is shaped to align with any site of a limb, a trunk, a neck, and a head in a human body or living body. The cover is disposed at least in a portion of an outer surface of the deformation portion so as to come into contact with any site of the body, and prevents heat transfer from the deformation portion to the site of the body.

According to the fixing tool of the present disclosure, the cover for preventing the heat transfer from the deformation portion to the site of the body is disposed in at least a portion of the outer surface of the deformation portion. Therefore, the heat transfer to a human body of a user can be prevented when the deformation portion is deformed, and an unpleasant feeling of the user can be prevented.

A fixing tool is disclosed comprising: a deformation portion that is brought into a deformable state by mixing two or more different materials with each other, and that is cured in a state where the deformation portion is shaped to align with any site of a limb, a trunk, a neck, and a head in a body, and wherein the deformation portion includes a material which is foamed by a polymerization initiator.

A method is disclosed of fixing a predetermined site of a human body, the method comprising: applying a force to a fixing tool, the fixing tool including a deformation portion with two or more different materials; mixing the two or more different materials with each other to bring at least one of the two or more different materials into an expansively deformable state; placing the predetermined site of the human body in contact with the fixing tool with the two or more different material in the expansively deformable state; and curing the two or more different material in a state where the deformation portion is shaped around at least a portion of the predetermined site of the human body

### BRIEF DESCRIPTION OF THE DRAWINGS

FIG. 1 is a schematic perspective view illustrating a fixing tool according to a first embodiment of the present disclosure in accordance with an exemplary embodiment.

FIG. 2 is a plan view of FIG. 1.

FIG. 3 is a perspective view illustrating the fixing tool having a contact portion obtained before the contact portion is deformed.

FIG. 4 is a plan view illustrating a state where a forearm is placed in the fixing tool having the contact portion obtained before the contact portion is deformed or when the contact portion is not completely deformed.

FIG. 5 is a cross-sectional view illustrating each structure of a deformation member and a heat insulating material, which configure the fixing tool.

FIG. 6 is a view for describing an example when the fixing tool is deformed, and is a cross-sectional view illustrating a state before the fixing tool is deformed.

FIG. 7 is a view for describing an example when the fixing tool is deformed, and is a cross-sectional view illustrating a state after the fixing tool is deformed.

FIG. 8 is an enlarged view illustrating a portion 8 of FIG. 2.

FIG. 9 is an enlarged view illustrating a modification example of FIG. 8.

FIG. 10 is a schematic perspective view illustrating an example when the fixing tool is used to fix a head and a neck, as a modification example of the first embodiment.

FIG. 11 is a schematic perspective view illustrating a fixing tool according to a second embodiment of the present disclosure.

FIG. 12 is a perspective view illustrating a state where a contact portion (deformation member and cover) and a restriction portion are disassembled in the fixing tool of FIG. 11.

FIG. 13 is a front view of FIG. 11.

FIG. 14 is a plan view of FIG. 11.

FIG. 15 is a cross-sectional view taken along line XV-XV in FIG. 14.

### DETAILED DESCRIPTION

Hereinafter, embodiments according to the present disclosure will be described with reference to the accompanying drawings. In describing the drawings, the same reference numerals will be given to the same elements, and repeated description will be omitted. In some cases, a size or proportion of members in the drawings may be exaggerated and different from an actual size or proportion for convenience of the description.

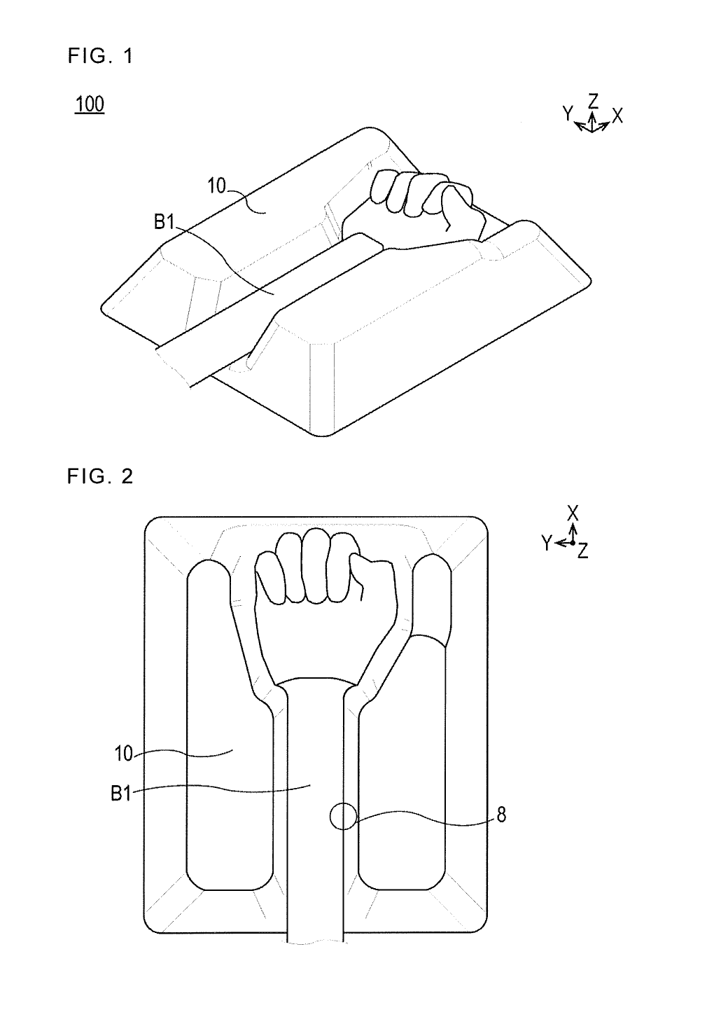

Hereinafter, a fixing tool 100 according to a first embodiment will be described with reference to FIGS. 1 to 9. FIGS. 1 to 9 are views for describing the fixing tool 100 according to the first embodiment of the present disclosure in accordance with an exemplary embodiment.

The fixing tool 100 according to the present embodiment can be used, for example, when fixing a forearm B1 or the like of a patient who is in a stable state on a hospital bed. Referring to FIG. 1, in brief, the fixing tool 100 has a contact portion 10, which includes a deformation member 11 and comes into contact with the forearm B1. Hereinafter, the fixing tool 100 will be described in detail.

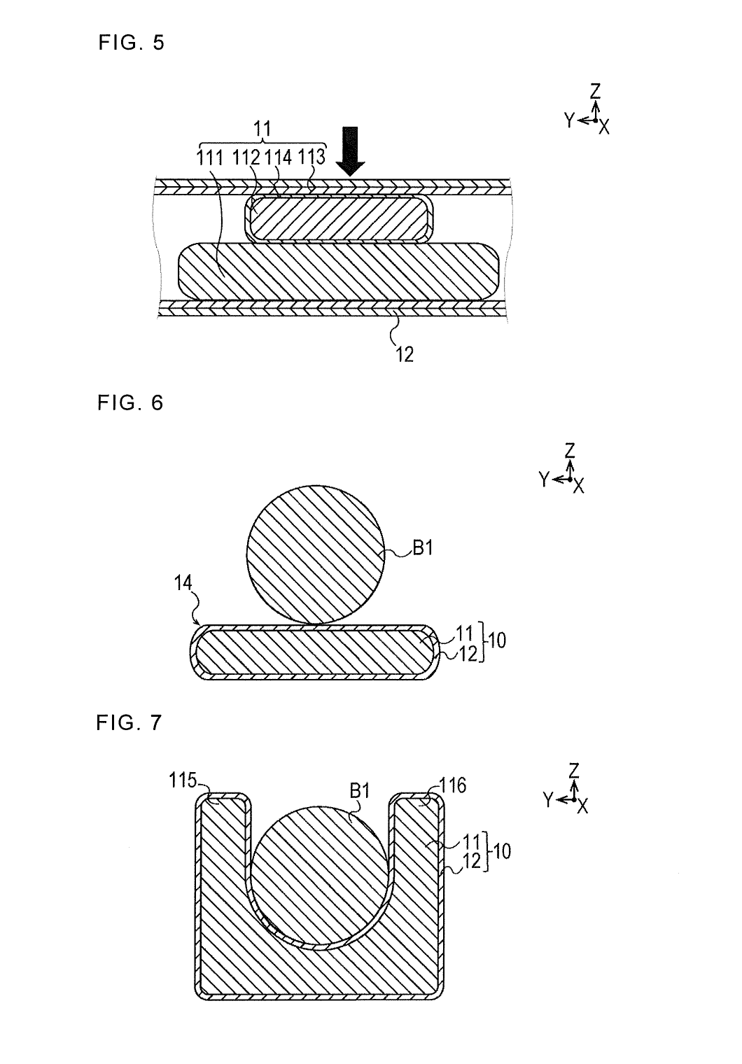

The contact portion 10 has the deformation member 11 (corresponding to a deformation portion) which is configured to be deformable as illustrated in FIGS. 5 to 7 and is cured in a state where the deformation member 11 is shaped to align with a site of the forearm B1, and a heat insulating material 12 (corresponding to a cover) disposed on an outer surface of the deformation member 11.

In accordance with an exemplary embodiment, the deformation member 11 is a site which is shaped to align with a shape of a desired site when the desired site of a human body such as the forearm B1 is fixed (supported) as illustrated in FIG. 1. The deformation member 11 has a first deforming agent 111 and a second deforming agent 112 whose states are changed by being mixed as illustrated in FIG. 5, and an accommodation member 113 and an accommodation member 114. The first deforming agent 111 and the second deforming agent 112 are configured to include liquids having mutually different types or the like. The first deforming agent 111 and the second deforming agent 112 are foamed by mixing two different liquids or the like with each other. In the present embodiment, a volume thereof increases while both of these are deformed from the liquid to a solid. For example, the first deforming agent 111 can be configured to contain polyol, and the second deforming agent 112 can be configured to contain polyisocyanate. Polyurethane can be obtained by mixing these materials with each other. However, as long as a deformable state is achieved by mixing both of these with each other and both of these are cured thereafter, the materials of the first deforming agent 111 and the second deforming agent 112 are not limited to the above-described configurations. The first deforming agent 111 is also called a foaming agent, and the second deforming agent 112 is also called a polymerization initiator.

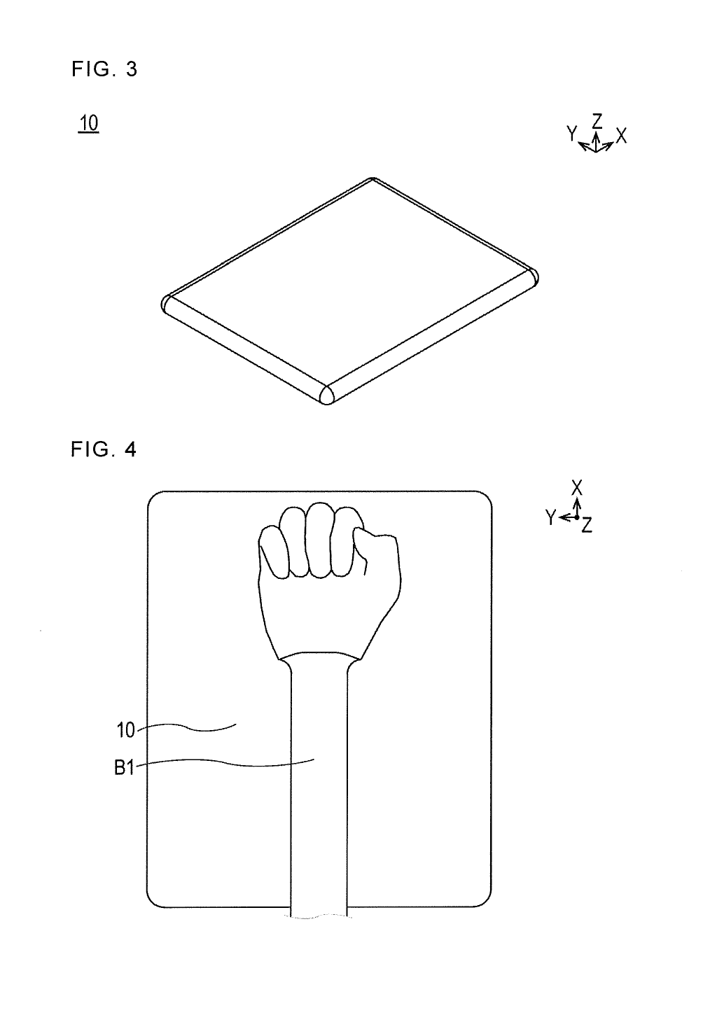

The accommodation member 113 is a member for accommodating the first deforming agent 111 and the second deforming agent 112 so as not to leak out. The accommodation member 113 is disposed on a surface of the deformation member 11, and forms an outer shape of the deformation member 11 and the contact portion 10. In order that the deformation member 11 can at least partially cover various sites of the human body as illustrated in FIGS. 3 and 6, the deformation member 11 can be formed in a so-called a substantially flat rectangular parallelepiped shape as a shape maintained before the first deforming agent 111 and the second deforming agent 112 are mixed with each other.

However, as long as the first deforming agent 111 and the second deforming agent 112 can be mixed with each other and can be shaped to align with the site of the forearm B1, a shape of the deformation member 11 before the first deforming agent 111 and the second deforming agent 112 are mixed with each other is not limited to the above-described shape. The shape of the deformation member 11 may be configured to be a substantially flat columnar shape in addition to the above-described shape.

The accommodation member 114 is a member disposed in an interior space of the accommodation member 113 as illustrated in FIG. 5 so that the second deforming agent 112 is accommodated to be isolated from the first deforming agent 111 until the second deforming agent 112 is mixed with the first deforming agent 111. Therefore, the accommodation member 114 may be thinner than the accommodation member 113, or may be configured so that the accommodation member 114 can be easily broken by an external impact by being provided with a slit (notch). In accordance with an exemplary embodiment, the accommodation members 113 and 114 are configured to include polyethylene, for example.

When coming into contact with the human body such as the forearm B1, the deformation member 11 is formed to have a substantially arc shape by being deformed as illustrated in FIG. 7, when viewed in a cross section orthogonal to a longitudinal direction of the forearm B1. In the description herein, the longitudinal direction in a predetermined site of the human body such as the forearm B1 to which the fixing tool 100 is attached is defined as a longitudinal direction X in the drawings. In addition, a direction extending along a line segment connecting end portions 115 and 116 of the deformation member 11 in a cross section orthogonal to the longitudinal direction X as illustrated in FIG. 7 is defined as a first transverse direction Y. In addition, a direction orthogonal to the longitudinal direction X and the first transverse direction Y is defined as a second transverse direction Z.

The deformation member 11 is shaped in a state of being aligned with a shape of the forearm B1 by mixing the first deforming agent 111 and the second deforming agent 112 with each other as described above and maintaining a state where the forearm B1 is brought into contact with the deformation member 11 for a predetermined period of time. In this state, the site such as the forearm B1 is fixed by the fixing tool 100 as illustrated in FIGS. 1 and 2. Here, “fixing” the predetermined site of the human body such as the forearm B1 described above means a state where the forearm B1 cannot move at least in the first transverse direction Y by placing the forearm B1 in the fixing tool 100 including the cured deformation member 11. As a measure of “fixing” in the above description, as illustrated in FIG. 7, an outer surface of the contact portion 10 is in contact with approximately half or more of an outer periphery of the forearm B1, or covers approximately the half or more.

In accordance with an exemplary embodiment, the heat insulating material 12 comes into contact with a fixing target site such as the forearm B1 and helps prevent heat transfer to a user such as a patient when heat is generated after the first deforming agent 111 and the second deforming agent 112 are mixed with each other. The heat insulating material 12 is configured to cover the entire periphery of the accommodation member 113 of the deformation member 11 in the present embodiment. However, as long as the heat insulating material 12 is provided in such as at least a place where both of the site the forearm B1 and the deformation member 11 have direct contact with each other when in use, the heat insulating material 12 does not necessarily cover the entire periphery of the accommodation member 113 of the deformation member 11. In accordance with an exemplary embodiment, the heat insulating material 12 can be configured to include heat insulating nonwoven fabric, for example, such as glass fiber-added vinyl chloride sheet, alumina oxide or glass fiber.

In addition, for example, the heat insulating material 12 can be configured to serve as a film-like member including polyethylene terephthalate or the like and having a concavo-convex shape formed on the surface by means of emboss processing. The heat insulating material 12 can also be configured to include those which are formed by molding a hollow ceramic fiber into a mesh shape in addition to the above-described member.

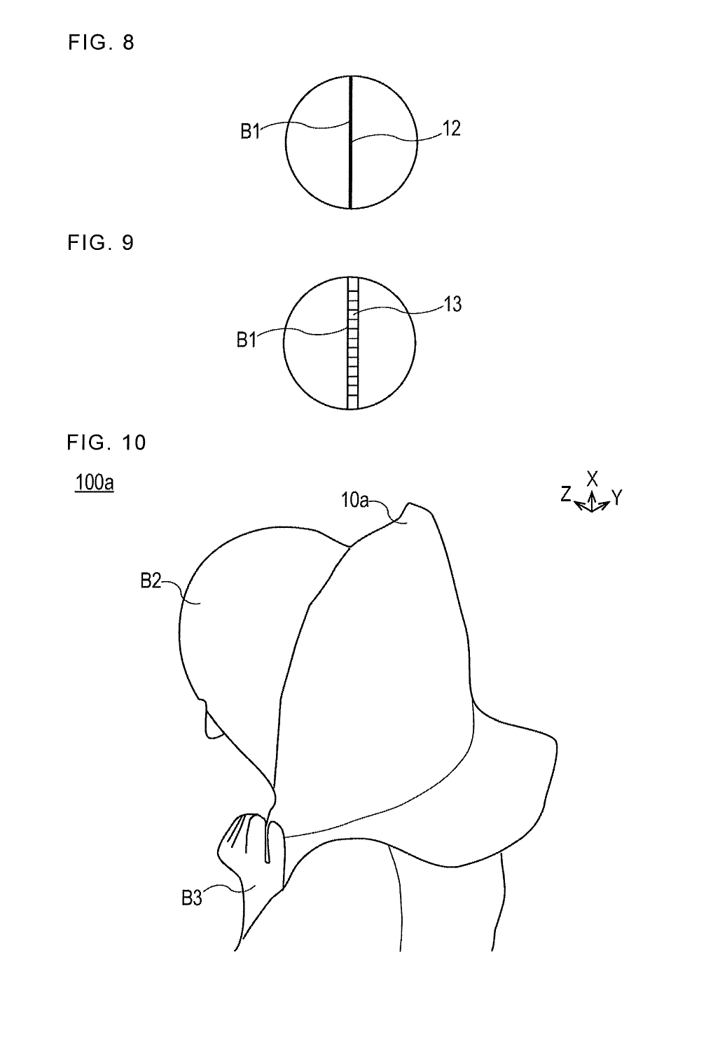

FIG. 8 is an enlarged view illustrating a portion 8 of FIG. 2, and FIG. 9 is an enlarged view illustrating a modification example of FIG. 8. In FIG. 8, the outer surface of the heat insulating material 12 comes into surface contact with an outer surface, a so-called skin of the forearm B1. In addition to the above-described configuration, the heat insulating material 12 can be configured to include elastomer, polyurethane, or vinyl chloride as in a modification example illustrated in FIG. 9. A plurality of flexible projections 13 molded in a convex shape can be disposed on the outer surface of the heat insulating material 12. In accordance with an exemplary embodiment, it is possible to reduce a contact area between the heat insulating material 12 and the forearm B1 by disposing the plurality of flexible projections 13 in a site in contact with the forearm B1 on the outer surface of the heat insulating material 12. In addition, heat generated when the deformation member 11 is foamed can be efficiently radiated outward from a place which is not in contact with the forearm B1 on the outer surface of the heat insulating material 12. Accordingly, heat transfer to a user can be further prevented.

Next, a method of using the fixing tool 100 according to the present embodiment will be described. Here, an example when a medical procedure (surgery) is performed on a patient in a medical field will be described.

For example, the fixing tool 100 is used when the forearm B1 or the like is fixed before the medical procedure is performed by introducing a catheter into the body of the patient.

When the fixing tool 100 is used, the fixing tool 100 is first prepared. The fixing tool 100 covers a portion of an outside surface (outer surface) of the forearm B1 as illustrated in FIGS. 4 and 6, and includes the deformation member 11 which is longer than the forearm B1 in the first transverse direction Y of the forearm B1.

Next, a pressing force is applied from the outside of the contact portion 10 to a site where the accommodation member 114 is disposed as illustrated by an arrow in FIG. 5 so as to break the accommodation member 114. The second deforming agent 112 accommodated in the accommodation member 114 is mixed with the first deforming agent 111. In this manner, the first deforming agent 111 starts to be foamed, and is brought into an expansively deformable state.

Next, as illustrated in FIG. 4, the forearm B1 serving as a fixing target site is disposed at an intermediate position in the first transverse direction Y of the deformation member 11 in the contact portion 10. Here, a surface in a state before the deformation member 11 is deformed is referred to as a base portion 14 (refer to FIG. 6). In FIG. 6, the base portion 14 is configured to be substantially flat, but the base portion 14 is not always necessary to be flat.

Next, while the expansive deformation of the first deforming agent 111 and the position of the forearm B1 is adjusted, the contact portion 10 including the deformation member 11 is shaped to align with the forearm B1. In this manner, as illustrated in FIG. 7, in the contact portion 10 including the deformation member 11, the deformation member 11 is expansively deformed. A place other than the place which is in contact with the forearm B1 in the base portion 14 illustrated in FIG. 6 protrudes in an upward direction in FIG. 7, that is, toward the second transverse direction Z crossing the plane of the base portion 14. In this manner, a range in contact with the outer surface of the forearm B1 through the contact portion 10 increases compared to that before the deformation, thereby fixing the forearm B1.

In accordance with an exemplary embodiment, a degree of solidification is determined by a mixing degree between the first deforming agent 111 and the second deforming agent 112. Therefore, an operator may appropriately disperse a solidified place to a whole place by stretching a mixture of the first deforming agent 111 and the second deforming agent 112 with fingers or the like to a place, which is not progressively solidified in the deformation member 11. If the deformation member 11 is shaped, the forearm B1 is brought into a fixed state by the fixing tool 100, and thereafter, a medical procedure such as catheter introduction can be performed.

When the first deforming agent 111 and the second deforming agent 112 are mixed with each other, heat is generated due to the progress of polymerization reaction. If the fixing tool is brought into contact with the body of the user during the medical procedure, the place generating the heat as described above comes into contact with the user. Thus, the user may sweat or may feel itching, in some cases. These lead to a possibility that the user may feel discomfort.

In view of this point, in the present embodiment, the heat insulating material 12 is disposed so as to cover the outer surface of the deformation member 11. Accordingly, it is possible to prevent the generated heat transfer to the user. Therefore, the discomfort by preventing sweating or itching which may occur when the fixing tool 100 is used can be minimized.

In addition, the deformation member 11 employs a foaming agent such as the first deforming agent 111 and a polymerization initiator such as the second deforming agent 112. Accordingly, the deformation member 11 can be deformed into various shapes so as to align with a physique of the user.

FIG. 10 is a perspective view illustrating a modification example of the fixing tool according to the first embodiment. In the first embodiment, an embodiment adopted when the fixing tool 100 fixes the forearm B1 has been described. However, the fixing tool can also be used for fixing sites other than the forearm B1 as follows.

As illustrated in FIG. 10, a fixing tool 100a includes a contact portion 10a which comes into contact with a head B2 and a neck. A shape of the contact portion 10a before being deformed is substantially the same as that of the contact portion 10 in FIG. 3. In addition, similarly to the contact portion 10 according to the first embodiment, the contact portion 10a has the same configurations of the deformation member 11 and the heat insulating material 12. Accordingly, the same reference numerals are used for the same configurations, and thus, detailed description will be omitted.

A method of using the fixing tool 100a is as follows. First, the fixing tool 100a is first prepared. The fixing tool 100a covers a portion of the head B2 and the neck in the first transverse direction Y, and includes the deformation member 11 which is longer than the head B2 and the neck. Next, the first deforming agent 111 and the second deforming agent 112 are mixed with each other, thereby bringing the first deforming agent 111 into an expansively deformable state.

Next, the fixing tool 100a is disposed at any position on the head B2, for example, near the rear head. The first deforming agent 111 is protruded from the base portion 14 of the deformation member 11. In this manner, a range where the contact portion 10a is in contact with the outer surface of the head B2 and the neck is increased compared to a range formed before the deformation. In this manner, the head B2 and the neck are fixed by the fixing tool 100a.

When the deformation member 11 is expansively deformed, the end portion of the fixing tool 100a may be gripped with a finger B3 as illustrated in FIG. 10 so that the deformation member 11 is not deformed to an unintended shape. In this way, the head B2 and the neck in addition to the forearm B1 can also be firmly fixed using the fixing tool 100a.

FIGS. 11 to 15 are views for describing a fixing tool according to a second embodiment. In the first embodiment, an embodiment for fixing the forearm B1 has been described, and in the modification example of the first embodiment, an embodiment for fixing the head B2 and the neck has been described. However, in addition to the above-described embodiments, the fixing tool can also be used in fixing a leg B4, for example. In the following description, the same reference numerals will be used for configurations the same as those according to the first embodiment.

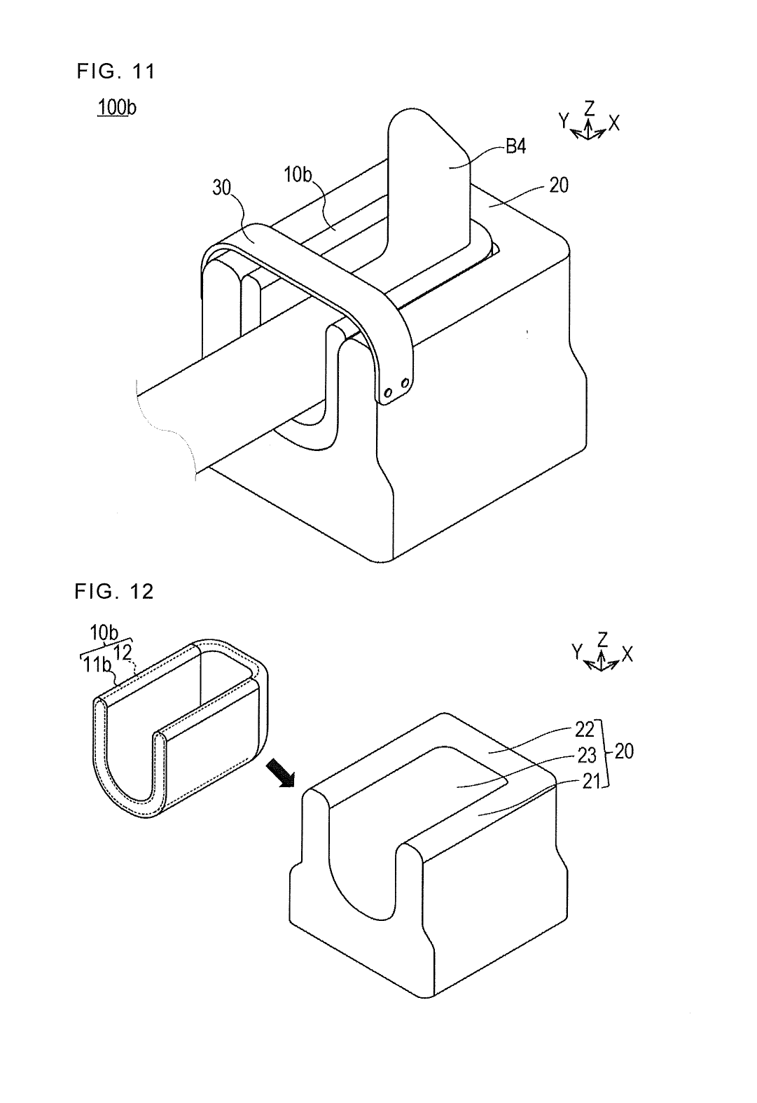

As illustrated in FIGS. 11 and 12, a fixing tool 100b according to a second embodiment has a contact portion 10b which includes a deformation member 11b and comes into contact with the leg B4, a restriction portion 20 which restricts a shape of the deformation member 11b, and a holding portion 30 which holds a state where the contact portion 10b is in contact with the leg B4.

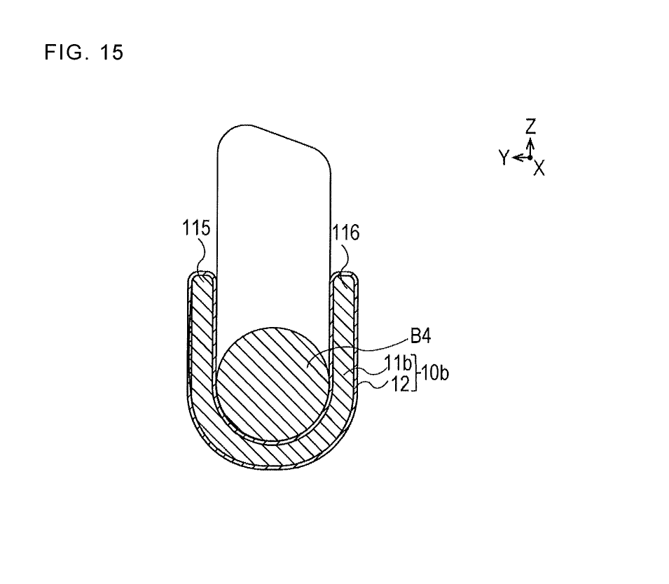

Similarly to the first embodiment, the contact portion 10b has the deformation member 11b and the heat insulating material 12. As illustrated in FIGS. 12 and 15, the deformation member 11b has an outer shape different from that of the deformation member 11 according to the first embodiment. However, the deformation member 11b has the same internal structure, and thus, detailed description will be omitted. The heat insulating material 12 is the same as that according to the first embodiment, and thus, detailed description will be omitted. The deformation member 11b of the contact portion 10b is formed in advance to align with the outer shape of the leg B4 so as to be easily shaped conforming to the leg B4 as illustrated in FIG. 12. However, without being limited thereto, a configuration may be adopted which has a flat rectangular parallelepiped shape as in FIG. 3 according to the first embodiment.

The restriction portion 20 restricts the deformation of the deformation member 11b so that the deformation member 11b is not deformed into an unintended shape due to the mixing between the first deforming agent 111 and the second deforming agent 112. The restriction portion 20 corresponds to a foundation, a substrate, or a base on which the contact portion 10b is placed. As illustrated in FIG. 12, the restriction portion 20 has a horizontally transverse portion 21, an end portion enclosing portion 22, and a recess portion 23. The restriction portion 20 is disposed further outward from the contact portion 10b with reference to a site such as the leg B4.

The horizontally transverse portion 21 is disposed radially outward from the contact portion 10b with reference to the human body such as the leg B4, and comes into contact with the contact portion 10b. The end portion enclosing portion 22 is disposed outward from the contact portion 10b in the vicinity of the heel corresponding to the end portion of the leg B4 in the longitudinal direction X orthogonal to the first transverse direction Y, and comes into contact with the contact portion 10b. The recess portion 23 is a site in which the contact portion 10b is placed. In the present embodiment, the recess portion 23 is formed in a shape close to a shape of the leg B4 by the horizontally transverse portion 21 and the end portion enclosing portion 22. However, the shape is not limited thereto as long as the leg B4 can be fixed. In addition to the above-described configuration, a configuration may be adopted which includes only the horizontally transverse portion 21. The restriction portion 20 can be configured to include a material such as polypropylene, for example.

In addition, the restriction portion 20 may have a through-hole (not illustrated) penetrating in each thickness direction of the horizontally transverse portion 21 and the end portion enclosing portion 22 in order to radiate the heat generated when the deformation member 11 b is foamed. In this case, an opening having a shape of an end portion of the through-hole of the restriction portion 20 is formed on each outer surface of the recess portion 23, the horizontally transverse portion 21, and the end portion enclosing portion 22. According to this configuration, when the leg B4 is fixed by the fixing tool 100b, the fixing tool 100b causes the heat insulating material 12 to block the heat generated when the deformation member 11b is foamed in the contact portion 10b which comes into contact with the leg B4. In addition, in a place which is not in contact with the leg B4 on the outer surface of the heat insulating material 12, the heat generated when the deformation member 11b is foamed can be efficiently radiated outward via a through-hole disposed in the recess portion 23. Accordingly, it is possible to further prevent the heat transfer to the user.

Furthermore, the surface of the recess portion 23 of the restriction portion 20 may be formed of a metal material. The metal material disposed on the surface of the recess portion 23 is not particularly limited as long as the material has high thermal conductivity. For example, copper, aluminum, magnesium, titanium, or stainless steel may be used.

According to this configuration, the fixing tool 100b can transfer the heat generated by the foaming of the deformation member 11b to the metal surface of the recess portion 23, in the site in contact with the surface of the recess portion 23 on the outer surface of the contact portion 10b. Therefore, the heat generated by the foaming of the deformation member 11b is absorbed by the metal material disposed on the surface of the recess portion 23. In this manner, the heat can be efficiently radiated outward, and it is possible to further prevent the heat transfer to the user. The metal material disposed on the surface of the recess portion 23 may be disposed over the entire surface of the recess portion 23 so as to align with the shape of the leg B4, or may be partially disposed.

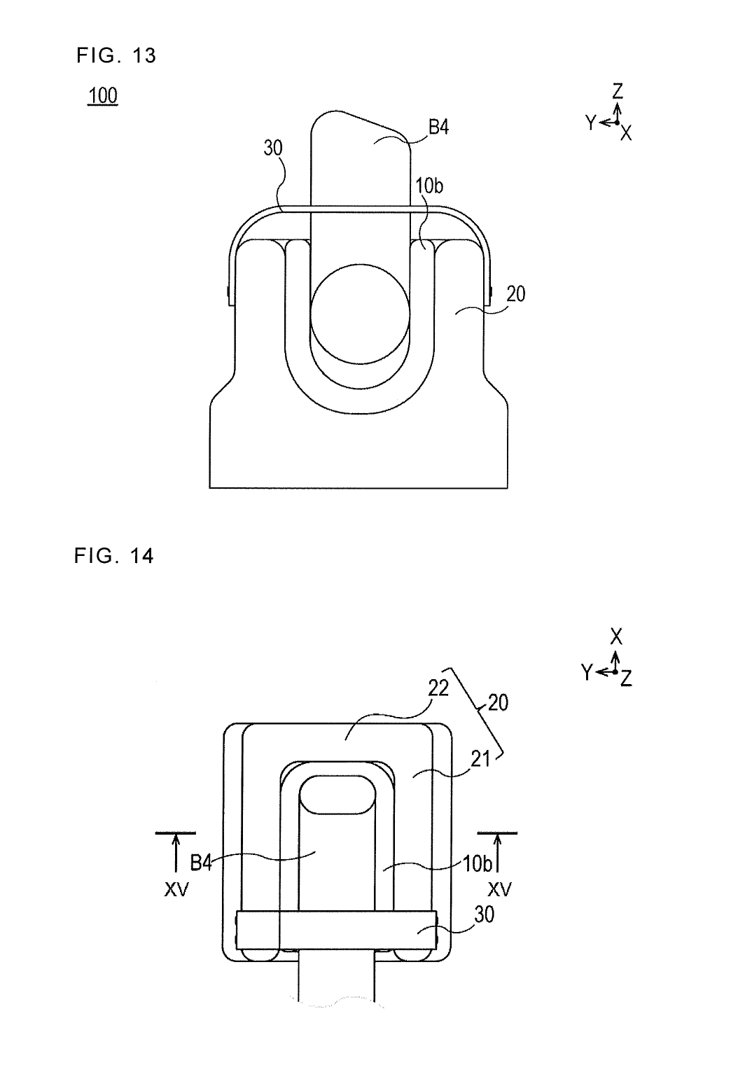

The holding portion 30 holds (maintains) a state where the heat insulating material 12 of the contact portion 10b is in contact with the leg B4. In accordance with an exemplary embodiment, the holding portion 30 is configured to include a cord, a band, or a strap-like member as illustrated in FIG.11, and is attached to the restriction portion 20. As illustrated in FIGS. 11 and 13, the holding portion 30 is disposed on a side substantially opposite to a side where the heat insulating material 12 of the contact portion 10b is in contact with the leg B4 with reference to the site such as the leg B4.

A method of using the fixing tool 100b according to the second embodiment is as follows. First, the contact portion 10b is prepared in a deflated state to align with a partial shape of the outer surface of the leg B4 as illustrated in FIG. 11. Next, the accommodation member 114 is broken so as to mix the second deforming agent 112 with the first deforming agent 111, thereby bringing the first deforming agent 111 into an expansively deformable state.

Next, the contact portion 10b is placed in the recess portion 23 of the restriction portion 20, and the leg B4 is set on the contact portion 10b. Next, the deformation member 11b is shaped to align with the leg B4. The second deforming agent 112 and the first deforming agent 111 may be mixed with each other after the contact portion 10b is placed in the recess portion 23.

In the present embodiment, the contact portion 10b including the deformation member 11b is placed in the recess portion 23 of the restriction portion 20, and the deformation member 11b is deformed in a state where the contact portion 10b is brought into contact with the leg B4. As described above, the recess portion 23 has a shape close to that of the leg B4. Therefore, the deformation member 11b is placed in the recess portion 23 so that the deformation member 11b can be easily shaped to align with the shape of the leg B4.

In addition, the fixing tool 100b includes the holding portion 30 which holds a state where the heat insulating material 12 is in contact with the leg B4 on the side opposite to the side with which the heat insulating material 12 of the contact portion 10b is in contact on the outer peripheral of the leg B4. In this manner, even in a case where the deformation member 11b is vigorously deformed, it is possible to prevent a disadvantage that the leg B4 is released from the contact with the heat insulating material 12.

The present embodiment is not limited to only the above-described embodiments, and various modifications can be made within the scope of claims. In the above-described embodiments, an embodiment using the fixing tool for the forearm B1, the head B2 and the neck, or the leg B4 has been described. However, the present disclosure is not limited thereto. In addition to the above-described embodiments, the fixing tool may be used for an upper arm, an abdomen, or a thigh.

In addition, an embodiment has been described in which the second deforming agent 112 for initiating the foaming is disposed inside the accommodation member 114 configuring the deformation member 11. However, the present disclosure is not limited thereto. In addition to the above-described configuration, a configuration may be adopted in which the second deforming agent 112 is injected from the outside of the deformation member 11 immediately before the foaming.

In addition, in the above-described embodiments, an embodiment has been described in which the first deforming agent 111 is changed from the liquid to the solid by mixing the first deforming agent 111 and the second deforming agent 112 with each other. However, the present disclosure is not limited thereto. In addition to the above-embodiments, a polymerization initiator may be mixed with a material such as silicon. In this manner, the material may be changed to a gel-like state having higher viscosity of the fluid compared to the viscosity before the material is mixed.

In addition, an embodiment has been described in which the first deforming agent 111 is accommodated inside the accommodation member 113 configuring the deformation member 11. However, the present disclosure is not limited thereto. On the assumption of a shape obtained after curing, a flow path for dispersing the first deforming agent 111 may be disposed inside the accommodation member 113.

In addition, in the above-described embodiments, an embodiment has been described in which the fixing tool 100 is used in the medical field when the catheter is introduced. However, the present disclosure is not limited thereto. In addition to the above-described embodiments, the fixing tool may be used when the head B2 or the like is fixed when a user is at home or when the user gets on a vehicle.

In addition, an embodiment has been described in which the fixing tools 100 and 100a according to the first embodiment and the modification example of the first embodiment have the contact portion 10 and does not include the restriction portion 20 and the holding portion 30 unlike the fixing tool 100b according to the second embodiment. However, the present disclosure is not limited thereto. In addition to the above-described embodiments, similarly to the fixing tool 100b according to the second embodiment, a configuration may be adopted which includes the restriction portion 20 and the holding portion 30.

The detailed description above describes a fixing tool. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A fixing tool comprising:
a deformation portion that is brought into a deformable state by mixing two or more different materials with each other, and that is cured in a state where the deformation portion is shaped to align with any site of a limb, a trunk, a neck, and a head in a body; and
a cover that is disposed in at least a portion of an outer surface of the deformation portion so as to come into contact with any site of the body, and that prevents heat transfer from the deformation portion to the site of the body.

2. The fixing tool according to claim 1, further comprising:
a restriction portion that includes a recess portion in which the deformation portion is placed, and that causes the deformation portion to be deformed to align with the site of the body, in a state where the deformation portion is placed in the recess portion and the site of the body is brought into contact with the deformation portion.

3. The fixing tool according to claim 1, further comprising:
a holding portion that is disposed on a side opposite to a side with which the cover comes into contact in the site of the body, and that holds a state where the cover is in contact with a portion on an outer periphery of the site of the body.

4. The fixing tool according to claim 1,
wherein the deformation portion includes a material which is foamed by a polymerization initiator.

5. The fixing tool according to claim 1, wherein the two or more different materials is a first deforming agent and a second deforming agent.

6. The fixing tool according to claim 5, wherein the first deforming agent and the second deforming agent are different liquids.

7. The fixing tool according to claim 5, wherein the first deforming agent changes from a liquid to a solid by mixing the first deforming agent with the second deforming agent.

8. The fixing tool according to claim 5, wherein the first deforming agent contains polyol, and the second deforming agent contains polyisocyanate.

9. The fixing tool according to claim 5, wherein the first deforming agent is a foaming agent, and the second deforming agent is a polymerization initiator.

10. The fixing tool according to claim 5, wherein the first deforming agent is a silicon and the second deforming agent is a polymerization initiator, and wherein the first deforming agent and the second deforming agent have a higher viscosity after mixing than before the mixing of the first deforming agent and the second deforming agent.

11. The fixing tool according to claim 5, wherein the deformation member has a form of a flat rectangular parallelepiped shape before the mixing of the first deforming agent and the second deforming agent.

12. A fixing tool comprising:
a deformation portion that is brought into a deformable state by mixing two or more different materials with each other, and that is cured in a state where the deformation portion is shaped to align with any site of a limb, a trunk, a neck, and a head in a body, and wherein the deformation portion includes a material which is foamed by a polymerization initiator.

13. The fixing tool according to claim 12, further comprising:
a restriction portion that includes a recess portion in which the deformation portion is placed, and that causes the deformation portion to be deformed to align with the site of the body, in a state where the deformation portion is placed in the recess portion and the site of the body is brought into contact with the deformation portion.

14. The fixing tool according to claim 12, further comprising:
a holding portion that is disposed on a side opposite to a side with which the cover comes into contact in the site of the body, and that holds a state where the cover is in contact with a portion on an outer periphery of the site of the body.

15. The fixing tool according to claim 12, wherein the two or more different materials is a first deforming agent and a second deforming agent, the second deforming agent being the a polymerization initiator.

16. The fixing tool according to claim 15, wherein the first deforming agent and the second deforming agent are different liquids, and wherein the first deforming agent changes from a liquid to a solid by mixing the first deforming agent with the second deforming agent.

17. The fixing tool according to claim 15, wherein the first deforming agent contains polyol, and the second deforming agent contains polyisocyanate.

18. A method of fixing a predetermined site of a human body, the method comprising:
applying a force to a fixing tool, the fixing tool including a deformation portion with two or more different materials;
mixing the two or more different materials with each other to bring at least one of the two or more different materials into an expansively deformable state;
placing the predetermined site of the human body in contact with the fixing tool with the two or more different material in the expansively deformable state; and
curing the two or more different material in a state where the deformation portion is shaped around at least a portion of the predetermined site of the human body.

19. The method of claim 18, further comprising:
preventing heat transfer from the deformation portion to the predetermined site of the human body by disposing at least a portion of an outer surface of the deformation portion, which comes into contact with the predetermined site of the human body in a heat insulating material.

20. The method of claim 18, further comprising:
performing a medical procedure after the curing of the two or more different materials on the predetermined site of the human body.
